(19) United States
(12) Patent Application Publication
(10) Pub. No.: US 2011/0002961 A1
(43) Pub. Date: Jan. 6, 2011

(54) Interleukin 12 paracrine gene delivery enhanced CTL immunotherapy

(76) Inventor: Paul Hermonat Hermonat, Little Rock, AR (US)

Correspondence Address: FUESS & DAVIDENAS, 6 Anthony Ln, Mabelvale, AR, 72103, US

(21) Appl. No.: 12/380,740

(22) Filed: Mar. 3, 2009

Related U.S. Application Data
(60) Provisional application No. 61/067,857, filed on Mar. 3, 2008.

(51) Int. Cl. A61K 39/21; A61K 39/12; A61K 39/145; A61K 39/29; A61K 39/245; A61K 39/275; A61K 39/235; A61K 39/02; A61K 39/002; A61P 35/00; A61P 37/02

(52) U.S. Cl. 424/208.1; 424/204.1; 424/207.1; 424/209.1; 424/227.1; 424/228.1; 424/229.1; 424/232.1; 424/233.1; 424/234.1; 424/265.1

(57) ABSTRACT

A method of treating a patient with a disease wherein the patient contains diseased cells which cells contain antigens for identification and which cells are capable of presenting at least part of said antigen on their surface by an HLA class I (or equivalent) molecule, the method comprising administering to the patient a therapeutically effective amount of either antigen presenting cells which have been gene delivered with the interleukin 12 (IL-12) gene plus a relevant antigen. In a related set of claims said method refers to the administration to a patient of therapeutic levels of cytotoxic T lymphocytes (CTL) which recognize at least part of said antigen when presented by an HLA class I (or equivalent) molecule on the surface of a cell and which these CTL were stimulated by the antigen presenting cells, mentioned in the first section, which have been gene delivered with the interleukin 12 (IL-12) gene plus a relevant antigen.

### FIELD OF THE INVENTION

The present invention generally concerns enhancing Immunotherapy using cytotoxic T lymphocytes.

### DESCRIPTION OF RELATED ART

Introduction:

Adaptive-immunogene therapy holds great promise for generating novel and effective anti-cancer treatments. One major approach has been the genetic manipulation of antigen presenting dendritic cells (DC) to manipulate them in such a manner that they effectively present specific antigens, delivered by gene therapy, for the efficient stimulation of cytotoxic T lymphocytes (CTL) (1). Various protocols for generating DC in vitro from peripheral blood monocytes have been developed and widely used. These new technologies permit the easy in vitro manipulation of DC for laboratory and clinical studies (2,3). These protocols have include pulsing DC with tumor antigen fragments, antigen peptides, defined tumor antigens, or with antigen genes by way of retrovirus and adenovirus vectors (4-13). While others have concentrated on using these other virus vectors for this task, we and a few others have concentrated on adeno-associated virus (AAV) (14,15), which is another vector useful for for gene delivery into hematopoietic progenitor cells (16-18), as well as antigen gene delivery into DC. We (19-26) and other groups (30-38) have found AAV to be very effective at DC transduction and that these resulting antigen-loaded DC are very effective at stimulating major histocompatibility complex (MHC) Class I-restricted, antigen specific CTL responses. While this technique has worked very well for generating effective CTL in vitro, it is recognized that there is significant resident tolerance to the tumors in patients and this tolerance must be overcome (39-41).

One additional gene therapy manipulation which could be undertaken to improve adaptive immune response is the delivery of Th1 response-associated cytokines. The superiority of cytokine gene therapy over exogenous cytokines, in regards to effectiveness of cytokine delivery, is obvious as most cytokines have extremely short half lives, ranging from a few minutes to a number of hours. The half life of some cytokines has been shown to be increased by fusion of the cytokine with longer-lived proteins, such as antibodies, antibody components, or albumin (42-46), or by conjugation of high molecular weight polymers of polyethylene glycol (PEG) (47,48). However, the delivery of the cytokine genes allows for the continuous expression of fresh, biologically active cytokine which may have two-three logarithms higher activity than commercial preparations on a per weight basis (19). If the most desirable CTL-enhancing, Th 1 response cytokine gene and target cell (DC or T cell) can be identified then this would increase the ability of immunotherapy to overpower the residence tolerance to the tumor.

Both the DC and CD4 helper T cells usually provide Th1-response cytokine support for promoting stimulation of CD8 CTL effector cells. The DC is a viable target for cytokine gene delivery as cytokines such as tumor neucrosis factor alpha (TNF″) clearly have effects on DC maturity and function. The T cell is also a viable target as the expression of certain cytokines, such as interferon gamma directly correlate with CTL killing ability. The use of gene therapy to IL-2 is one potentially useful Th1 response cytokine for immunogene therapy. IL2 is an essential factor for T cell expansion, proliferation and thus is important for the generation of CTL (49,50). IL-2 stimulates the production of certain cytokines such as IFN (which are tumoricidal at the site of metastasis. The IL-2 receptor (IL-2R) beta and gamma chains are able to activate the Janus family of tyrosine kinases (eg. Jak1 and Jak3). However for generating anti-cancer CTL IL-2 also has a “negative side”, that is it is needed for the maintenance of peripheral CD4+CD25+ regulatory T (TReg) cells, Tregs are known as down regulators, eliminators of self-reactive T cells (51,52). Perhaps, the most important negative effect of IL-2 on T cells is that it is associated with activation induced cell death (AICD)(53,54). This can result in the death of as much as 95% of the activated T cells.

IL-12 is yet another potentially useful cytokine for gene therapy protocols. Kuge et al. (1995) found that IL-12 induced rapid proliferation of CTL, peaking at days 4-5 post-addition (55). Mehrota et al. (1993) found that IL-12 augmented killing efficiency of CTL cells by 10-20 fold on a per cell basis (56). In addition they reported IL-12 also resulted in higher CTL numbers. They further determined that the IL-12 enhancing activity did not involve regulation of IL-2, but was independent of IL-2. There have been numerous studies on the generating of whole cell vaccines by the delivery of the IL-12 gene into tumor cells. Thus, taken together, the evidence suggests that IL-12 is a central cytokine in CTL response. In any case, we can use adeno-associated virus type 2 (AAV)-based gene delivery we can force the expression of these important cytokines into nearly whatever cell type we want or need. Thus, here we compared the delivery of interleukins (IL)-2 and -12 into DC or into T cells versus the addition of exogenous cytokine for their ability to generate robust CTL.

Materials and Methods:

Cells. HEK293, K562, SW480 colorectal adenocarcinoma cell line, Lncap-FGC prostate cancer line, Hs578T breast cancer cell line and H2126 lung cancer cell line were obtained from The American Culture Collection (ATCC), Mask cell line (??). EBV-transformed B cells (LCL) derived from five healthy donors were cultured in RPMI 1640 medium supplemented with 10% fetal bovine serum (FBS). The peripheral blood mononuclear cells (PBMC) from five healthy donors were separated by routine Ficoll gradient method. All blood donors were given informed consent in writing, and the study protocol conformed to the ethical guidelines of the 1975 Declaration of Helsinki as reflected in a priori approval by our Human Research Internal Review Board. The HLA haplotype of all donors were compatible with SW480 cells (HLA A2).

Construction of recombinant AAV vectors. Human CEA, IL-2, IL-12 p35 and IL-12 p40 cDNA were amplified by reverse-transcription polymerase chain reaction (RT-PCR), respectively. Trizol reagent (Invitrogen) was employed to isolate total mRNA from SW480 cells and normal human T lymphocytes, respectively. Then the total mRNA was separate from the total RNA using Oligotex mRNA isolation kit (Qiagen). After the first-strand cDNA was generated, PCR amplification for each of the cDNA was carried out using the following primer pair: CEA; 5′-ACCATGGAGTCTCCCTCG-3′ and 5′-CTATATCAGAGCAAC CCC-3′ that amplify the sequence from nucleotides 112 to 2223 (1). IL-2: 5′-TGCCACAATGT CACCAGTAT-3′ and 5′-CGGAGTATACTTATATGACGC-3′ that amplify the sequence from nucleotides 41 to 553 (57). IL-12 p35: 5′-AATGTGGCCCCCTGGGTC-3′ and 5′-TTTAGGAAGCA TTCAGAT-3′ that amplify the sequence from nucleotides 215 to 978 (3). IIL-12 p40: 5′-AGATGT GTCACCAGCAGTTG-3′ and 5′-AACCTAACTGCAGGGCAC-3′ that amplify the sequence from nucleotides 41 to 1032 (3). All of cDNA were sequenced and determined to be identical to the published sequence (57-59). AAV/CEA and AAV/IL-2 vectors were constructed as the previously described (16, 20 ???). IL-12 p35 and p40 cDNA were inserted in the downstream of p5 and CMV early promoter of a AAV vector, respectively.

Generation of recombinant AAV virus. The plasmid pSH3 can express AAV type 2 rep and cap genes and adenovirus E2A, VA1 and E4 genes to allow rAAV DNA replication and packaging into viral particles without contaminating wild type AAV and adenovirus (37). The recombinant AAV (rAAV) vectors were co-lipofected into HK 293 cells with plasmidpSH3 (60). The rAAV were harvested by routine method after 4 days (18-27). To generate the purified rAAV the one-step column purification technique described by Auricchio, et al. was used (38). The rAAV were tittered as described previously by dot blot hybridization (18-27).

Generation of DC infected by recombinant AAV. After the PBMC (5×10⁶) were cultured for two hours with AIM-V medium, the non-adherent cells were removed. The monocytes (Mo) were infected immediately with 1×10⁸ encapsidated genomes (eg)/ml of AAV/CEA virus, AAV/CEA plus AAV/IL-2 virus or AAV/CEA plus AAV/IL-12. After four hours the medium/virus solution was removed and the cells were finally fed with the medium containing recombinant human GM-CSF (Immunex, 800 IU/ml). At day 2 and 4, to induce the maturation of Mo into DC, recombinant human IL-4 and TNF-” (R & D SYSTEMS.) at 1000 IU/ml and 20 ng/ml were added to the medium, respectively. The AAV/CEA-infected Mo were also treated without or with exogenous human recombinant IL-2 (20 IU/ml) or IL-12 (20 ng/ml). The medium and cytokines were replaced every two days. Finally, at day 6 the DC were mixed with CD3+ T cells.

Infection of CD3+ T cells with rAAV. Pan T Cell Isolation Kit II (Miltenyi Biotec) was employed to isolate CD3+ T cells from the non-adherent cells from the PBMC according to the kit instruction. At day 5 the CD3+ T cells (1×10⁶) were infected with 1×10⁸ eg/ml of AAV/IL-2 or AAV/IL-12 virus. Only AAV/IL-2-infected T cells were cultured without AIM-V medium containing 20 IU/ml. The exogenous IL-12-treated CD3+ T cells were cultured as a control (20 ng/ml).

Analysis of rAAV chromosomal integration. The total DNA were isolated from the rAAV-infected or uninfected DC or T cells using DNAzol reagent (Invitrogen) according to supplier's protocol. Chromosomal integration of the AAV/CEA genome studies by vector-chromosome junction PCR amplification and southern blot analysis, as previously described (18-27).

RT-PCR experession analysis of transduced DC or T cells for CEA, IL-2, and IL-12 expression. CEA, IL-2 and IL-12 mRNA expression was detected by RT-PCR. Isolation and amplification of mRNA and cellular mRNA control and IL-2 and IL-12 p40 primer pairs are described above. CEA was designed following primer pair: 5′-CTCCTGCTCACAC CCTCACT-3′ and nt 5′-CGTTG GAGTTG TTGCTGGTG-3′ that amplify the sequence from nucleotides 166 to 1114.

Analysis of transduction and expression of CEA antigen and the cytokines by FACS. At day 6 of Mo/DC culture and day 4 of rAAV infection of CD3+ T cells, intracellular staining assay of Pala et al (2000) was employed, respectively, to analyze the expression and efficient transduction of rAAV according to the routine method (61). After the rAAV-infected or control cells were harvested the cells were fixed and permeabilized. The cells were incubated with the FITC- or PE-labeled monoclonal antibodies recognizing the following antigens, respectively: CEA, IL-2, IL-10 plus IL-12 (BD Pharmingen), Control irrelevant isotype-matched FITC- or PE-conjugated monoclonal antibodies were also obtained from BD Pharmingen. A FACSCalibur flow cytometer (Becton-Dickinson) was used for data acquisitions. At least ten thousand events were counted for each sample.

Analysis of IL-10 and IL-12 protein expression in the DC. Expression level of IL-10 and IL-12 in DC were analyzed with intracellular staining assay described above. In addition, Human IL-12 p70 and IL-10 were measured in the DC supernatants by enzyme-linked immunosorbent assay (ELISA) using commercially available kits (Biosource International). The sensitivity of the IL-10 and IL-12 ELISA is <1 pg/ml and <0.5 pg/ml, respectively. At day 6 IL-10 and IL-12 p70 secretion was measured in duplicate according to the manufacturer's instructions. Table 1

Cell surface marker analysis of DC. For the analysis of DC a panel of FITC- or PE-labeled monoclonal antibodies recognizing the following antigens was used: CD14, CD40 (Chemicon International), HLA-DR, CD80, CD8, CD86 and isotype-matched antibodies (BD Pharmingen). After 6 days the non-adherent DC were harvested (>95% viable as assessed by Trypan blue exclusion) the cells counted and distributed. Stained cells were assayed for surface markers according to the routine method. Table 2

Generation of cytotoxic T lymphocytes (CTL). At day 6 of DC culture the mature DC were harvested and mixed with CD3+ T lymphocytes (ratios from 20:1, T: DC), respectively. For the AAV/IL-2 plus AAV/CEA-infected DC, exogenous IL-2-treated plus AAVCEA-infected DC and AAV/IL-2-infected CD3+ T cells, the mixtures were only cultured with recombinant human IL-7 (20 ng/ml). Other mixtures were cultured in AIM-V containing recombinant human IL-2 (20 IU/ml) and IL-7 (20 ng/ml). The exogenous IL-12-treated CD3+ T cell-DC mixtures were still cultured in addition of IL-12 (20 ng/ml). The medium and cytokines were replaced every two days. After 8 days post-priming the cells were harvested and analyzed further.

Analysis of T cell proliferation stimulated by rAAV-infected DC. After the CD3+ T cells were mixed with the DC at day 6, each group of the mixed cells were inoculated into 5 wells of 96-well cell culture plates, respectively. There were 5H10⁵ cells (200 :l) in each well. After the mixed cells were cultured for 8 hours in 37 EC, 5% CO₂, ³H-TDR incorporation test was carried out according to the routine method.

CD marker analysis of activated T cell Populations. For the analysis of activated T cells, at day 8 of the mixed cell culture the primed T cell populations were analyzed for their surface markers with immunofluorescence staining by FACS. A panel of FITC- or PE-labeled monoclonal antibodies recognizing the following antigens was used: CD4, CD8, CD25 and CD69 (BD Pharmingen).

Analysis for the level of IFN-(in the activated T cell populations. At day 8 days post-priming T cells were harvested. The intracellular staining assay was performed to analyze the expression of IFN-(in the T cells according to the method described above. FITC-labeled anti-IFN-(monoclonal antibody (BD Pharmingen) was used.

Analysis of CEA-specific and MHC Class I-restricted CTL killing activity. 6-hour chromium-51 (⁵¹Cr) release assay (18-27) was used to analyze the killing activity of CTL elicited by AAV/CEA-infected and control DC against the target cells. After The CTL cells and ⁵¹Cr-labeled target cells were mixed (20:1) and incubated for 6 hours at 37° C. with 5% CO₂. To determine the structures on the target cells involved in lysis, mouse anti-HLA Class I monoclonal antibodies were used to block cytotoxicity. The ⁵¹Cr-labeled targets were pre-incubated with mouse anti-human MHC class I antibody (Serotec) for 1 hr. before the ⁵¹Cr release assay was performed. The mouse anti-human MHC class II antibody (Serotec) was also used as a control. K562 cells were used as targets to observe natural killer (NK) cell activity and a series of cells (without CEA expression) were used as negative controls. LCL derived from different donors were infected by AAV/CEA/Neo virus, and cultured in the medium plus 50 :g/ml of G418 for more than 15 days to generate CEA expressing LCL cells.

### RESULTS

Cloning of CEA, IL-2 and IL-12 and Delivery into DC and T Cells.

The generation of CTL requires at least effective, antigen-loaded DC, CD4+ T helper cells, and CD8+ effector precursor cells. The interplay between these cell types requires physical interactions between both the CD4+ and CD8+ T cells and the antigen presenting DC. But this interplay also involves the stimulation of all of these cell types by important cytokines whose mechanism's of action have not yet been fully determined. In any case many TH1 response-associated cytokines will likely be useful in immuno-gene therapy protocols once their optimal use (and mechanism of action) are determined. Furthermore, regarding antigenic targets for therapy against cancer, chorio-embryonic antigen (CEA) may be very useful for stimulating Th1 response against adeno-carcinomas.

To develop working gene therapy vectors for delivering these agents the CEA antigen gene was cloned from breast cancer cell line Hs578T XXX and ligated upstream of the cytomegalovirus immediate early promoter (or AAV p5 promoter?) within a fully gutted AAV2 based vector (d13-97). IL-2 (cloned from peripheral blood lymphocytes cells) and IL-12 (obtained from Invivo Gen) were also cloned into AAV in a similar manner. RAAV were generated in the usual manner using the helper plasmid pSH3 (60) and titered by standard dot blot assay as descrbed previously (18-27). The titers of the AAV/CEA, AAV/IL2, and AAV/IL12 virus ranged from 9-10×10¹¹ (data not shown). These virus were used to determine their efficiency of gene delivery into DC and T cells. Actually, our technique for transducing DC is to infect precursor freshly adherent peripheral blood monocytes with rAAV, to treat these cells with GM-CSF alone for two days, then add IL4 to induce their differentiation into DC (2,3). This technique has proven to be very effective in generating specific antigen-present presenting DC (18-27) and cytokine-expressing DC (19).

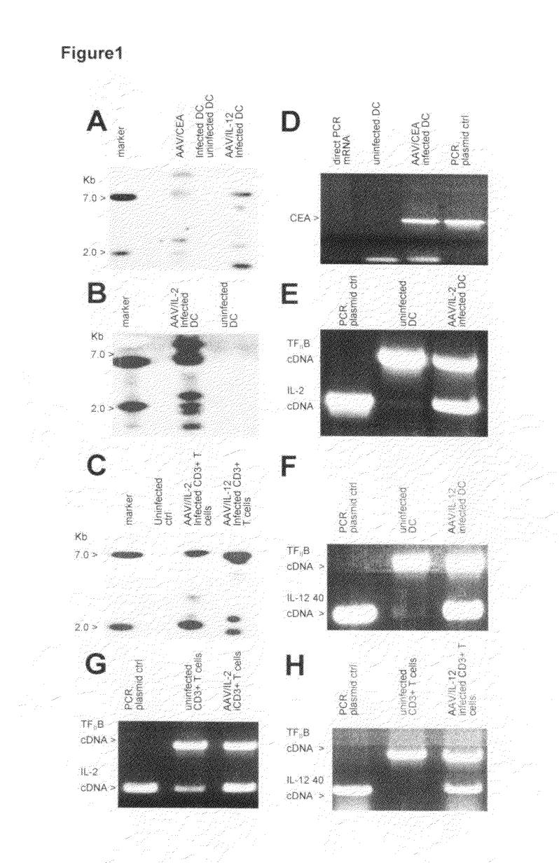

rAAV provirual chromosomal integration and expression of CEA, IL-2 and IL-12. One issue in the field of AAV-based gene therapy is the form of AAV latency within these transduced primary cells. In tissue culture transduced cell lines often display a chromosomally integrated provirus, whle in vivo transduced cells often show the latent rAAV DNA as an episomal element. To address this issue for rAAV transduction of DC and T cells the chromosomal DNA of transduced cells was analyzed for integrated provirus by PCR amplification of vector-chromosome junctions by using one primer directed towards the vector and anotherdirected towards the AluI repetitive chromosomal element. In this experiment we amplify vector chromosomal junctions in cells where rAAV has chromosomally integrated close to an Alu I repetitive element (one PCR primer directed against vector sequences and another against Alu I repetitive element). While inefficient, as it will only identify provirus integrated immediately adjacent to AluI elements in a specific orientation. As shown in FIG. 1A through 1C, this technique clearly demonstrates some level of chromosomal latency in DC and CD3+ T cells by the rAAV DNA integration.

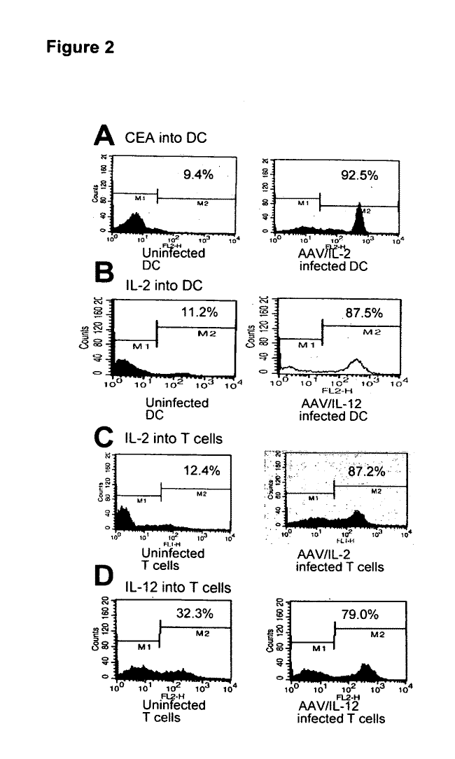

Furthermore, it is important that rAAV provirus express their transgene. in FIG. 1D through 1H it is shown that the resulting rAAV provirus transcriptionally express their respective transgenes by RT-PCR analysis, in both DC and T cells. To observe both the transduction efficiency and protein expression of the CEA, IL-2, and IL12 proteins we carried out an intracellular staining analysis of tansduced and untransduced DC and T cells. The transduction efficiency of DC by AAV/CEA and AAV/IL-2, as shown in FIGS. 2A and 2B, was approximately 90%. This agrees with our earlier studies with other transgenes. The transduction efficency of CD3+ T cells, as shown in FIGS. 2C and D, was approximately 79-87%. Thus transduction efficiency using AAV 2 was high for both the DC and T cells.

Characterization of Transduced DC.

Our goal is to generate the most robust anti-cancer CTL possible so as to overpower resident tolerance of the tumor micro-environment. Both IL2 and IL12 are Th1-response associated cytokines and their gene expression, either delivered by a paracrine approach into DC or an autocrine-approach into T cells might stimulate a higher level of proliferation of the effector cells than the standard delivery of exogenous cytokine into the cell culture. However, CTL response “robust-ness” incorporates, in our view, at least two major attributes, first is the level of responder T cell proliferation and second is the level of killing by these resulting CTL on a per cell basis. It is unclear if paracrine versus autocrine cytokine expression is an important issue, at least for IL2 and IL12. If a cytokine were found to be effective in an autocrine approach this would allow those genetically CTL to be somewhat more independent from DC and helper T cells in their continued proliferation and reactivity. At least this is what we envisioned.

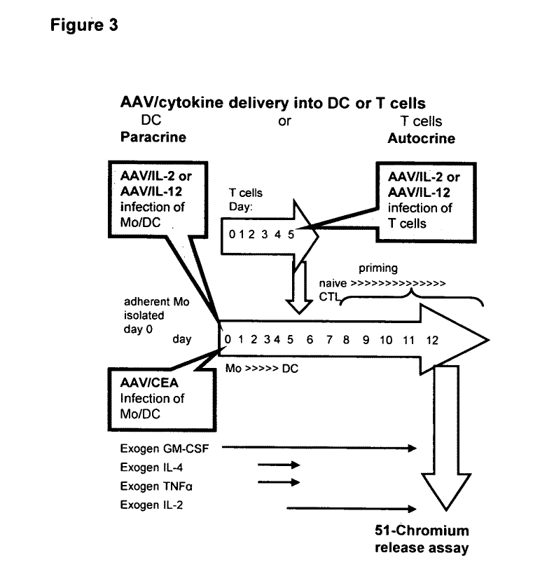

The structure of the experiment is shown in FIG. 3, where we either infected DC with the AAV/cytokine vector at day 0, or the T cells on day 5 just before their addition to the CEA antigen loaded DC. DC were always loaded by infection with AAV/CEA on day 0.

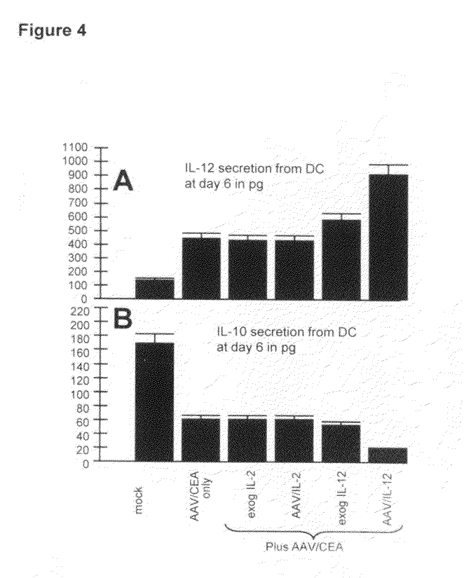

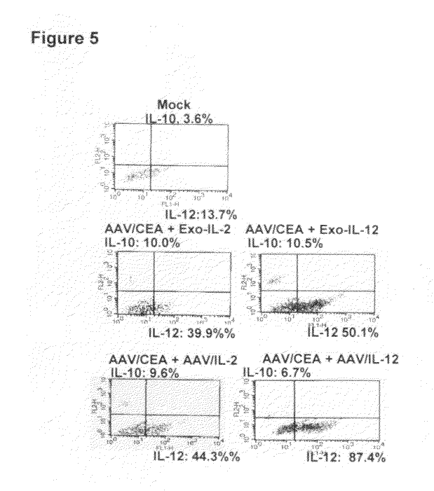

We examined the DC on day 6, as shown in Table 1, for surface expression of CD14, CD40, CD80, CD83, and CD 86 by FACS and found that CD80, CD86, and CD83 were up-regulated by rAAV infection as shown previously (18-27). The addition of either exogenous IL-12 or AAV/IL-12 further up-regulated these markers, with the use of AAV/IL-12 having the more profound effect. Most importantly CD80 and CD86 were expressed at very high levels. We further observed the expression level of IL-12 and IL-10 by DC by these various treatments. DC were either treated with exogenous IL-2 or IL12, or AAV/IL-2 or AAV/IL-12. IL-12 is a TH 1 response cytokine and IL-10 is a TH 2 cytokine so higher IL-12/IL-10 ratios reflect, the likelihood that these DC would stimulate a more robust TH 1 CTL response. As shown in FIG. 4, the simple delivery of the antigen by rAAV was enough to dramatically increase the IL12/IL10 secretion ratio over mock treated DC. The addition of exogenous IL-2 or AAV/IL-2 had no significant effect on improving this ratio. In contrast to IL-2, the addition of exogenous IL-12 did improve the IL-12:IL-10 secretion ratio (IL-12 has a half life of 5-10 hours). However, the transduction of the IL-12 gene by rAAV dramatically increased the IL-12:IL-10 secretion ratio by DC above all other treatments. We further analyzed these DC to investigate what percentage of these cells were involved in the secretion of these cytokines by observing intracellular cytokine by intracellular staining. FIG. 5, shows that, consistent with the levels of secreted cytokine, the AAV/IL-12 treated DC had the highest percentage of cells actively producing IL-12 and the lowest producing IL-10. These data suggest that the AAV/IL-12 treatment resulted in the most TH1 response-promoting DC. However, these data also suggest there may be a positive feedback loop involved in IL-12 expression as suggested by the increased secretion of IL-12 upon the addition of exogenous IL-12. These data also suggest that IL-12 may actively down-regulate IL-10 expression in DC.

Characterization of Stimulated T Cells.

We then turned our attention to the T cells generated by these various treatments. A robust TH1 CTL response is usually consistent with a high CD8:CD4 ratio. The resulting cell population stimulated by the various DC treatments was analyzed by FACS and the results listed in Table 2. As can be seen all T cell populations generated by AAV-transduced DC had a high CD8 to CD4 ratios, indicating a robust TH1 response, consistent with the higher levels of CD80 and CD86 expressed by AAV-infected DC. However T cells derived from AAV/IL-12-treated DC had the highest CD8/CD4 ratios, consistent these cells having high CD80, CD83, and CD86 expression, and lowest IL-10 expression. It is also noteworthy that the IL-2 treated DC, either exogenous or AAV-based IL-2, had higher CD4+ cells than CD8+. The IFN(:IL-4 ratios in the T cell population were also analyzed as a more direct measurement of the robustness of the TH1 response.

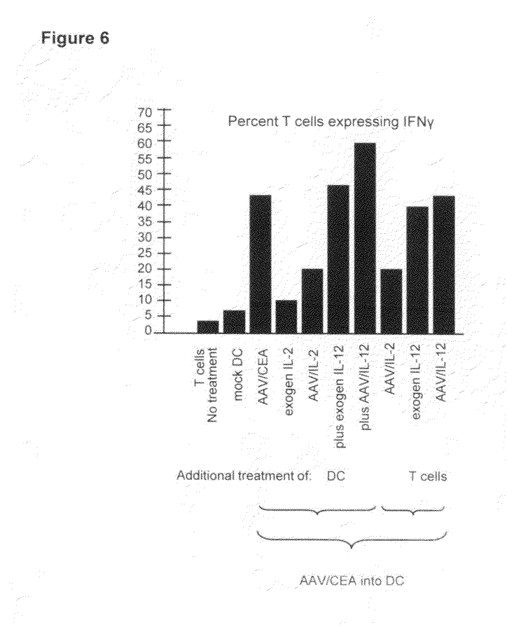

CD3 cells were also isolated from the general population and analyzed for IFN(expression. IFN(is another TH 1 response cytokine expressed by T cells, and many laboratories use IFN(expression by T cells as a substitute for evaluating the level of CTL response and as a predictor of CTL killing capability (62). FIG. 6 shows the percentage of T cells which express IFN(as determined by intracellular staining. These data contain some noteworthy findings. First, the treatment of DC with exogenous IL-2, or with AAV/IL-2, or the direct treatment of T cells with AAV/IL-2 resulted in a lowering of IFN(expression in the T cell population compared to control T cells (CTL) generated by AAV/CEA-loaded DC alone. Thus the use of IL-2, in any form, was detrimental to IFN(production. Second, exogenous IL-12 treatment of DC or T cells, while not inhibiting IFN(percent expression as did IL-2, did not enhance IFN(expression either. Third, only the treatment of DC with AAV/IL-12 resulted in percent T cell IFN(expression. These data suggest that the gene delivery of IL-12 only by a paracrine approach, into DC, can significantly enhance IFN(expression in the resulting CTL population. Autocrine delivery of AAV/IL-2 hurts IFN(expression and AAV/IL-12 autocrine delivery offers no advantage. These are surprising results as, to our knowledge, such paracrine “favoritism” for IL-12 or autocrine inhibition of IFN(production by IL-2 has never been reported previously.

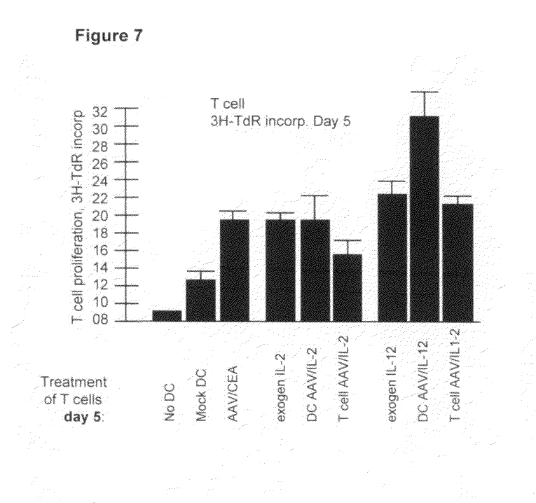

The generation of responder CTL involves both the proliferation of CD4+ helper T cells as well as proliferation of the CD8+ T cells themselves. To test the level of T cell proliferation we carried out the standard protocol for the generation of antigen-specific CTL. However, in addition loading the DC with the antigen (AAV/CEA) we also added the delivery of AAV/IL2 or AAV/IL12 into DC or T cells. Proliferation of CD3+ T cells was measured by the incorporation of 3H-TdR, and the resuts are shown in FIG. 7. Two different levels of proliferation stand out in this experiment. First, delivery of IL-2 directly into T cells resulted in lower levels of T cell proliferation, suggesting a favoritism for IL-2 paracrine delivery (into DC). In sharp contrast, the paracrine delivery of AAV/IL-12 resulted in very high T cell proliferation. However, unlike AAV/IL-2 into DC,which showed no increase in T cell proliferation over exogenous IL-2, T cell proliferation resulting from AAV/IL-12 into DC was much higher than the use of exogenous IL-12 cytokine. We expected that these levels of T cell proliferation would predict the level of target cell killing by these proliferating T cells (CTL).

The are only limited studies on the delivery of cytokine genes into T cells so we further analyzed these cells for changes in known important parameters by autocrine IL-2 and IL-12 gene delivery. The CD8/CD4 ratio is one important attribute already discussed and this ratio was not so different, as shown in Table 3, between autocrine IL-2 and IL-12 gene delivery or by exogenous IL-12 treatment. CD4+/CD25+ T regulator cells are also critical as they are involved in suppressing TH1 response. As shown in table 3 both autocrine IL-12 gene delivery and exogenous IL-12 treatments gave T cell populations with low levels of T regs. This is consistent with the higher IFN(expression in these T cell populations, and should be consistent with higher killing ability. CD8+/CD69+ early activated T effector cells are another important cell type as these cells are a significant part of the CTL population, and likely actively involved in target killing. As shown in table 3 both autocrine IL-12 gene delivery and exogenous IL-12 treatments gave T cell populations with high levels of CD69_ cells. This is also consistent with the higher IFN(expression in these T cell populations, and should also be consistent with higher killing ability.

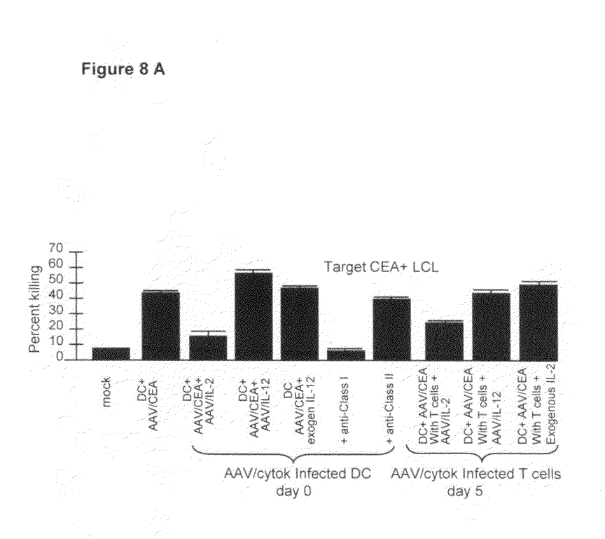

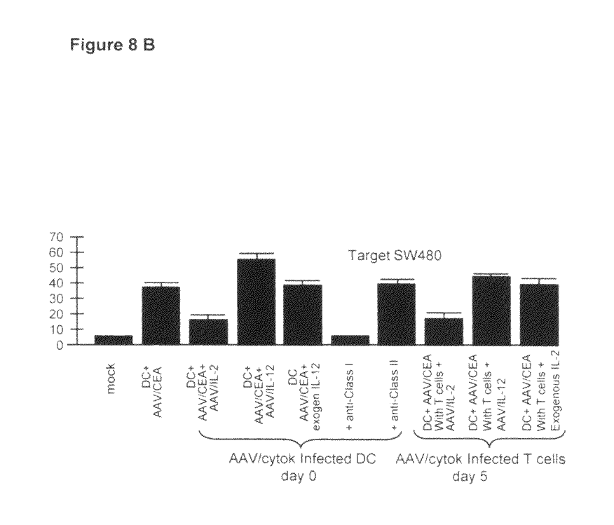

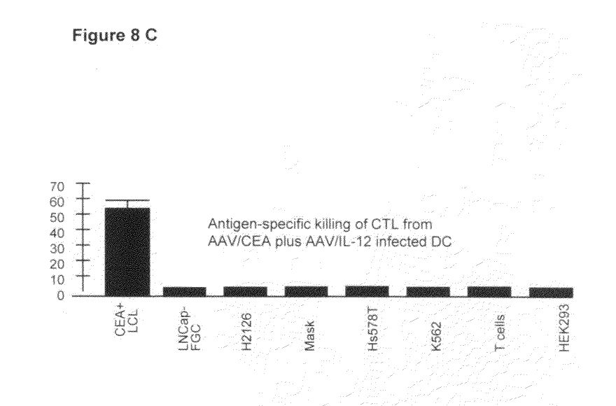

Effects of paracrine and autocrine IL-2 and IL-12 gene delivery CTL killing. Having characterized the AAV/cytokine transduced DC and T cells we then assayed the resulting CTL for their ability to kill a CEA-positive lymphoblastoid cell line (LCL) which was HLA-matched with blood donors. To carry out the testing of CTL killing do this we carried out the experiment depicted in FIG. 3, and tested for target killing in then stadard ⁵¹Cr release assay, and the results are shown in FIG. 8A. As can be seen the highest level of CEA-directed killing results from AAV/IL-12 paracrine delivery into DC. The high killing results from this particular treatment is fully consistent with the highest IL-12 secretion/production and lowest IL10 secretion/production in FIGS. 4 and 5, the highest IFN(production by T cells in FIG. 6, and the stimulation of the highest level of proliferation of T cells in FIG. 7. Also consistent with the other data the delivery of AAV/IL-2 into either DC or T cells resulted in an inhibition of CTL killing. Finally AAV/IL-12 delivery into T cells gave no advantage. Next, these same set of CTL were tested for killing of CEA-positive SW480 cells, as shown in FIG. 8B, and essentially identical killing resulted. Finally, these most effective CTL killers, those produced from AAV/CEA- and AAV/IL-12-treated DC, were tested against a variety of targets and demonstrated significant killing only against CEA-positive LCL, fully consistent with CEA-antigen specific killing.

### DISCUSSION

Our protocol using AAV/antigen-loaded DC to generate robust antigen-specific CTL appears superior others, being able to generate significant CTL with significant killing activity in only one stimulation. However, these assays are carried out in vitro and these CTL, when adoptively transferred into patients, will have to function within the tolerizing environment of the tumor. Thus one of our goals is to generate responder CTL which are self-sustaining, with higher killing, better proliferation, higher survival, and are able to maintain killing in the face of tolerizing tumor environment. One obvious way to improve CTL dedication and performance is to deliver certain Th 1 response cytokine genes into T cells (or DC). Certain cytokines have profound effects upon both T cells and DC function and survival. DC generation and function and Th1 (Type 1) response (CTL) can be promoted with appropriate cytokines. Cytokines are involved not only in the activation of CTL but also the proliferation of CTL, the survival and maintenance of CTL, and the protection of CTL from tolerizing agents. This latter is particularly important as studies show that as tumor burden increases the tumor-associated T cells show much killing ability. Furthermore, microenvironmental and continuous cytokine secretion may give superior results compared to the addition of exogenous cytokine proteins. Freshly secreted cytokines have a much higher biological activity than commercially available, recombinant cytokines used for patient injection (19). As we intend to carry out adoptive immunotherapy (injection of anti-cancer CTL) these experiments might directly translate into clinical therapies.

One important finding of this study this study is the total lack of help by IL-2 gene delivery in CTL generation either by the autocrine or paracrine strategy. IL-2 is very well studied and much of what is known might suggest help in TH1 response. IL-2 is expressed largely by activated T cells and modulates T cell function by activating, through its receptor, transcription factor STAT5 and possibly others (8). This signaling can occur in either a paracrine or an autocrine manner. However the details through which these two signaling modes operate during in vivo T cell responses is presently unknown. IL-2 was initially believed to be critical for T cell proliferation (63). The phenotype of IL-2-knockout mice subsequently indicated that IL-2 is not an essential component of T cell priming in vivo (64), however IL-2 does have certain influence on Ag-driven T cell responses in vivo (65,66) and is important for regulatory T cell (CD4+, CD25+) maintenance and function (67-69). IL-2 was believed to regulate both the T cells that produce IL-2 (autocrine signaling) as well as adjacent T cells (paracrine signaling) (70). Unfortunately the roles of autocrine vs paracrine IL-2 signaling in regulating T cell responses in vivo remains largely undetermined. It has been shown that CD8+ cell expansion in response to viruses can be either inhibited or enhanced by autocrine and paracrine IL-2 signaling, respectively (66), although there is no understanding of the mechanisms by which these two IL-2 signaling modes operate. To our surprise our gene delivery data shows that the paracrine route was the superior approach for IL-2. IL-2 gene delivery was best in enhancing target cell killing (FIG. 8) when transduced into DC, the paracrine route. These data are in fact consistent with the previous study on IL-2's paracrine and autocrine effects (66). Furthermore, we found that when introduced into T cells AAV/IL2 resulted in a T cell population with lowering killing abilities, also consistent with that previous study (66). The analysis of the cells in FIG. 7 suggests that the delivery of IL-2 into T cells resulted in the selective proliferation of T reg cells, which in turn inhibited the generation of CTL. IL-12 showed a somewhat similar preference, only the delivery of IL-12 into DC generated CTL with higher killing ability than into T cells or to the use of exogenous IL

In contrast with IL-2, IL-12 gene delivery did significantly help CTL killing, at least by the paracrine approach. This is the major finding of this study. The action of IL-12 gene delivery may also be more understandable. It is known that IL-12 promotes development of cell-mediated immune responses (71,72). Presently it is unclear the cells and mechanisms responsible for IL-12 production and of the cellular targets at the site of T cell priming. Some evidence suggests that appropriate stimuli can induce DC to release IL-12 and that this cytokine then acts on adjacent bystander T cells that recognize antigenic epitopes on adjacent and physically distinct DC (73). How this happens is unclear. In any case IL-12 appears to function in a paracrine fashion, as IL-12 production and epitope presentation can be carried out by different cells. In addition, IL-12 production by DC is induced by the interaction between CD40 on DC and CD40 ligand expressed on T cells after activation (74). CD40L is an important and potent stimulus in up-regulating costimulatory molecules (75) and cytokines including IL-12 (76) and others. Others have directly shown that IL-12 primes DC in vitro for more effective presentation of a poorly immunogenic tumor peptides (77-79) and anti-bodies against IL-12 block this stimulation (80). Thus it could be argued that the predominant effect of IL-12 is on the DC and its ability to stimulate responder T cells as opposed to a direct role on the CTL themselves. Furthermore, there is some evidence that IL-12 directly into T cells might be problematicfor TH1 responses, through its ability to sometimes induce expression of IL-10, a cytokine usually associated with lower TH1 response (81-82).

Enhancing TH1 response by delivery cytokines through gene therapy would seem to be a straight forward goal as genes can continuously generate fresh cytokine whose effects are limited in part because of their short half-lives. However, this study shows there are complexities to this approach. Appropriate TH1 response cytokines (eg. IL-12) must be delivered into the appropriate cell type for improving CTL killing. We have found in this study that IL-12 functioned better when delivered into the DC which then stimulate the T cells and provided the cytokine in a paracrine situation. To our knowledge this is the first time this “cytokine autocrine-paracrine geme delivery tropism” has been described. It was very surprisingly that the delivery of IL-2 in T cells or DC actually hurt CTL-killing efficiency. This knowledge regarding cytokine gene delivery “appropriateness” is important. While there is evidence in the literature for IL-2 and IL-12 activities which might explain these cytokine delivery tropisms, these issues require further research to fully understand, and optimize cytokine gene delivery. In conclusion, we consider these data to suggest that AAV/cytokine gene delivery, inparticular AAV/IL-12 delivery into DC, may have utility, but only when the appropriate cell is targeted. A “global” mechanism or approach for cytokine gene delivery does not seem plausible. Likely each Th1 response cytokine must be analyzed individually as to their best target cell type, their best route of delivery.

### REFERENCES

1) Steinman, R. A. The dendritic cell system and its role in immunogenicity. Annu. Rev. Imm. 1991. 9: 271-296.

2) Sallusto, F., and Lanzavecchia, A. Efficient presentation of soluble antigen by cultured human dendritic cells is maintained by granulocyte/macrophage colony-stimulating factor plus interleukin 4 and downregulated by tumor necrosis factor alpha. J. Exper. Med. 1994. 179(4): 1109-1118.

3) Romani. N., Gruner, S., Brang, D., Kampgen, E., Lenz, A., Trockenbacher, B., Konwalinka, G., Fritsch, P. O., Steinman, R. M., and Schuler, G. Proliferating dendritic cell progenitors in human blood. J. Exper. Med. 1994. 180: 83-93.

4) Young, J. W., and Inaba, K. Dendritic cells as adjuvants for class I major histocompatibility complex-restricted antitumor immunity. J. Exp. Med. 1996. 183: 7-11.

5) Zivotgel, L., Mayordomo, J. I., Tjandrawan, T., Delao, A. B., Clarke, M. R., Lotze, M. T., and Storkus, W. J. Therapy of murine tumors with tumor peptide-pulsed dendritic cells: dependence on T cells, B7 costimulationm and T helper cell I-associated cytokines. J. Exp. Med. 1996. 183: 87-97.

6) Paglia, P., Chiodoni, C., Rodolfo, M., and Colombo, M. P. Murine dendritic cells loaded in vitro with soluable protein prime cytotoxic T lymphocytes against tumor antigen in vivo. J. Exp. Med. 1996. 183; 317-322.

7) Alexander, M., Salgaller, M., Leseban, C., Barnes, W. A., Rosenberg, S. A., and Steller, M. A. Generation of tumor-specific cytotoxic T lymphocytes from peripheral blood of cervical cancer patients by in vitro stimulation with a synthetic human papillomavirus type 16 E7 epitope. Am. J. Obstet. Gynecol. 1996. 175: 1586-1593.

8) Philip, R., Brunette, E., Ashton, J., Alters, S., Gadea, J., Sorich, M., Yau, J., O'donoghue, G., Lebkowski, J., Okarma, T., and Philip, M. Transgene expression in dendritic cells to induce antigen-specific cytotoxic T cells in healthy donors. Cancer Gene Therapy. 1998. 5: 236-246.

9) Mcarthur, J. G., and Mulligan, R. C. Induction of protective anti-tumor immunity by gene-modified dendritic cells. J. Immunotherapy. 1998. 21: 41-47.

10) Sonderbye. L., Feng, S., Yacoubian, S., Buehler, H., Ahsan, N., Mulligan, R., and Langhoff, E. In vivo and in vitro modulation of immune stimulatory capacity of primary dendritic cells by adenovirus-mediated gene transduction. Exper. & Clin. lmmunogenet. 1998. 15: 100-111.

11) Kim, C. J., Prevette, T., Cormier, J., Overwijk, W., Roden, M., Restifo, N. P., Rosenberg, S. A., and Marincola, F. M. Dendritic cells infected with poxviruses encoding MART-1/Melan A sensitize T lymphocytes in vitro. J. Immunother. 1997. 20: 276-286.

12) Santin, A. D., Hermonat, P. L., Ravaggi, A., Chiriva-internati, M., Hiserodt, J. C., Pecorelli, S., Parham, G. P., and Cannon, M. Generation of an MHC class I restricted cytotoxic T cell response against autologous cervical cancer tumor cells by lipofection of synthetic human papillomavirus types 16 and 18 E7 protein into primary dendritic cells. J. Virol. 1999. 73: 5402-5410.

13) Santin, A. D., Hermonat, P. L., Ravaggi, A., Cowen, C., Coke, C., Pecorelli, S., Cannon, M. J., and Parham, G. P. Development, characterization, and distribution of adoptively transferred peripheral blood lymphocytes primed by human papillomavirus E7. European J. Gynaecologic Oncology. 2000. 21: 17-23.

14) Hermonat, P. L., and Muzyczka, N. Use of adeno-associated virus as a mammalian DNA cloning vector: transduction of neomycin resistance into mammalian tissue culture cells. PNAS U.S.A. 81: 6466-6470, 1984.

15) Tratschin J D, West M H, Sandbank T, Carter B J. A human parvovirus, adeno-associated virus, as a eucaryotic vector: transient expression and encapsidation of the procaryotic gene for chloramphenicol acetyltransferase. Mol Cell Biol. 1984 October; 4(10):2072-81.

16) Laface, D., Hermonat, P. L., Wakeland, E. K., and Peck, A. B. Gene transfer into hematopoietic progenitor cells mediated by an adeno-associated virus vector. Virology 162: 483-486, 1988.

17) Zhou, S. Z., Broxmeyer, H. E, Cooper, S., Harrington, M. A., and Srivastava, A. Adeno-associated virus 2-mediated gene transfer in murine hematopoietic progenitor cells. Experimental Hematology 21: 928-933, 1993.

18) Fisher-Adams, . . . Chatterjee, S. Integration of adeno-associated virus vectors in CD34+human hematopoietic progenitor cells after transduction. Blood. 88: 492-504, 1996.

19) Liu, Y., Santin, A. D., Mane, M., Chiriva-internati, M., Parham, G. P., Ravaggi, A. and Hermonat, P. L. Transduction and utility of the granulocyte macrophage-colony stimulating factor gene into Monocytes and dendritic cells by adeno-associated virus. J. Inf. Cytok. Res. 20: 21-30, 2000.

20) Chiriva-Internati, M., Liu, Y., . . . and Hermonat, P. L. Efficient generation of cytotoxic T lymphocytes against cervical cancer cells by adeno-associated virus/human papillomavirus type 16 E7 antigen gene transduction into dendritic cells. Europ. J. Imm. 32: 30-8, 2002.

21) Liu, Y., Chiriva-Internati, M., Grizzi, F., Salati, E., Roman, J. J., Lim, S., and Hermonat P L. Rapid induction of cytotoxic T-cell response against cervical cancer cells by human papillomavirus type 16 E6 antigen gene delivery into human dendritic cells by an adeno-associated virus vector. Cancer Gene Therapy 8: 948-57, 2001.

22) Chiriva-Internati, M. Liu, Y., Lim, S., Grizzi, F. Zhou, W., Barlogie, B., Mehta, J. L., and Hermonat, P. L. (2003) Stringent testing of recombinant adeno-associated virus-gene loading of dendritic cells for generating cytotoxic T lymphocytes against a prototype self-antigen, multiple myeloma HM1.24. Blood 102: 3100-3107

23) Liu, Y., Chiriva-Internati, M., Luo, R., You, C. X., Mehta, J. L., Klimberg, V. S., and Hermonat, P. L. (2004) Generation of milk protein BA46/lactadherin-specific cytotoxic T lymphocytes by AAV/BA46 gene delivery into dendritic cells. Cancer Gene Therapy 12:304-312.

24) Chiriva-Internati M, Liu Y, Wan W, Tedesco A, Grizzi F, Cobos E, Frezza E E, and Hermonat P L. Recombinant Adeno-Associated Virus Expressing Sperm Protein 17 into Dendritic Cells Induce a Better Cancer Vaccine by Improving Generation of Anti-Sperm Protein 17-Specific Cytotoxic T-Lymphocytes. Immunology 2004 Vol 1; pp 75-80.” Cytokine Network Regulation and Apoptosis” Monduzzi. Editore, 2004.(book chapter)

25) Liu Y, Zhou W, You C, Zheng H, You H, Liu H, Zhang D, Luo R, Kay H H, Hermonat P L. (2006) An autoimmune domain-reduced HCV core gene remains effective in stimulating anti-core cytotoxic T lymphocyte activity. Vaccine. Vaccine. 2006 Sep. 11; 24(37-39):6173-4.

26) You, H., Liu, Y., . . . Hermonat, P. L. (2006) HBV genes induce cytotoxic T lymphocyte response upon adeno-associated virus (AAV) vector delivery into dendritic cells. J. Viral Hepatitis 13(9):605-12

27) Prasad, C. K., Liu, Y., You, C., Luo, R., Mehta, J. L. and Hermonat, P. L. (2007) Generation, comparison of cytotoxic T lymphocyte stimulation against Her2/neu by rAAV and protein antigen loading of dendritic cells. Book chapter in Cancer and Gene Therapy, Research Signpost, Kerala, India, Editor: Paul L. Hermonat, in press (book chapter) Cancer and Gene Therapy

28) You, C., Liu, Y., Luo, R., You, H. Hermonat, P. L., and Mahadevan, M. (2007) Immunotherapy using cytotoxic T lymphocytes against prostate specific membrane antigen for prostate cancer. Book chapter in Cancer and Gene Therapy, Research Signpost, Kerala, India, Editor: Paul L. Hermonat, in press (book chapter) Cancer and Gene Therapy

29) Mahadevan, M., Liu, Y., Luo, R C, You, C X, You, H, Hermonat, P L Generation of robust cytotoxic T lymphocytes against prostate specific antigen by transduction of dendritic cells using protein and recombinant adeno associated virus. In press Cancer Immunology and Immunotherapy 56(10):1615-24.

30) Sun J Y, Senitzer D, Forman S J, Chatterjee S, Wong K K Jr. Related Articles, Identification of new MHC-restriction elements for presentation of the p210(BCR-ABL) fusion region to human cytotoxic T lymphocytes. Cancer Immunol Immunother. 2003 December; 52(12):761-70.

31) Sun J Y, Krouse R S, Forman S J, Senitzer D, Sniecinski I, Chatterjee S, Wong K K Jr. Related Articles, Immunogenicity of a p210(BCR-ABL) fusion domain candidate DNA vaccine targeted to dendritic cells by a recombinant adeno-associated virus vector in vitro. Cancer Res. 2002 Jun. 1; 62(11):3175-83

32) Ponnazhagan S. Mahendra G. Curiel D T. Shaw D R. Adeno-associated virus type 2-mediated transduction of human monocyte-derived dendritic cells: implications for ex vivo immunotherapy. Journal of Virology. 75(19):9493-501, 2001

33) You H, Cong M, Wang P, Yan Z Y, Xu Y, Lu Y, Wang B E, Jia J D. Transduction of dendritic cells by recombinant adeno-associated virus (rAAV) carrying hepatitis B virus antigens] Zhonghua Shi Yan He Lin Chuang Bing Du Xue Za Zhi. 2007 June; 21(2):105-7.

34) Cong M. Wang P. Yan Z Y. Tang S Z. Wang B E. Jia J D. Liu Y. You H. [Recombinant adeno-associated virus carrying hepatitis B virus C gene (rAAV-HBV-C) effectively pulses dendritic cells (DC) without affecting their maturity]. Chung Hua Kan Tsang Ping Tsa Chih. 13(1):17-9, 2005 January.

35) Xin K Q, Mizukami H, Urabe M, Toda Y, Shinoda K, Yoshida A, Oomura K, Kojima Y, Ichino M, Klinman D, Ozawa K, Okuda K. Induction of robust immune responses against human immunodeficiency virus is supported by the inherent tropism of adeno-associated virus type 5 for dendritic cells. J Virol. 2006 December; 80(24):11899-910.

36) Veron P, Allo V, Riviere C, Bernard J, Douar A M, Masurier C. Major subsets of human dendritic cells are efficiently transduced by self-complementary adeno-associated virus vectors 1 and 2. J Virol. 2007 May; 81(10):5385-94. Epub 2007

37) Aldrich W A, Ren C, White A F, Zhou S Z, Kumar S, Jenkins C B, Shaw D R, Strong T V, Triozzi P L, Ponnazhagan S. Enhanced transduction of mouse bone marrow-derived dendritic cells by repetitive infection with self-complementary adeno-associated virus 6 combined with immunostimulatory ligands. Gene Ther. 2006 January; 13(1):29-39.

38) Wong, K. K., Chatterjee, S. Recombinant Adeno-associated Virus Vectors for Leukemia Vaccine Development. Research Signpost, Kerala, India, Editor: Paul L. Hermonat, in press (book chapter) Cancer and Gene Therapy

39) Zou, W. Immunosuppressive networks in the tumour environment and their therapeutic relevance. Nature Rev. Cancer 5, 263-274 (2005).

40) Dunn, G. P., Bruce, A. T., Ikeda, H., Old, L. J. & Schreiber, R. D. Cancer immunoediting: from immunosurveillance to tumor escape. Nature Immunol. 3, 991-998 (2002).

41) Curiel, T. J. et al. Specific recruitment of regulatory T cells in ovarian carcinoma fosters immune privilege and predicts reduced survival. Nature Med. 10, 942-949 (2004).

42) Penichet M L, Morrison S L. Antibody-cytokine fusion proteins for the therapy of cancer. J Immunol Methods. 2001 Feb. 1; 248(1-2):91-101.

43) Gillies S D, Lan Y, Brunkhorst B, Wong W K, Li Y, Lo K M. Bi-functional cytokine fusion proteins for gene therapy and antibody-targeted treatment of cancer. Cancer Immunol Immunother. 2002 October; 51(8):449-60. Epub 2002 Jul. 12.

44) Balan V, Nelson D R, Sulkowski M S, Everson G T, Lambiase L R, Wiesner R H, Dickson R C, Post A B, Redfield R R, Davis G L, Neumann A U, Osborn B L, Freimuth W W, Subramanian G M. A Phase I/II study evaluating escalating doses of recombinant human albumin-interferon-alpha fusion protein in chronic hepatitis C patients who have failed previous interferon-alpha-based therapy. Antivir Ther. 2006; 11(1):35-45.

45) Melder R J, Osborn B L, Riccobene T, Kanakaraj P, Wei P, Chen G, Stolow D, Halpern W G, Migone T S, Wang Q, Grzegorzewski K J, Gallant G. “Pharmacokinetics and in vitro and in vivo anti-tumor response of an interleukin-2-human serum albumin fusion protein in mice. Cancer Immunol Immunother. 2005 June; 54(6):535-47.

46) Wills R J. Soike K F. Pharmacokinetics of human recombinant interferon-alpha I after i.v. infusion and im injection in African green monkeys. [Journal Article] Journal of Interferon Research. 8(4):427-32, 1988

47) Glue P, Fang J W, Rouzier-Panis R, Raffanel C, Sabo R, Gupta S K, Salfi M, Jacobs S. Pegylated interferon-alpha2b: pharmacokinetics, pharmacodynamics, safety, and preliminary efficacy data. Hepatitis C Intervention Therapy Group. Clin Pharmacol Ther. 2000 November; 68(5):556-67.

48) Shechter Y, Preciado-Patt L, Schreiber G, Fridkin M. “Prolonging the half-life of human interferon-alpha 2 in circulation: Design, preparation, and analysis of (2-sulfo-9-fluorenylmethoxycarbonyl)7-interferon-alpha 2. Proc Natl Acad Sci USA. 2001 Jan. 30; 98(3):1212-7.

49) He X. Tsang T C. Luo P. Zhang T. Harris D T. (2003) Enhanced tumor immunogenicity through coupling cytokine expression with antigen presentation. Cancer Gene Therapy. 10(9):669-77.

50) Wiryana P. Bui T. Faltynek C R. Ho R J. (1997) Augmentation of cell-mediated immunotherapy against herpes simplex virus by interleukins: comparison of in vivo effects of IL-2 and IL-7 on adoptively transferred T cells. Vaccine. 15(5):561-3.

51) Maloy, K. J. & Powrie, F. Fueling regulation: IL-2 keeps CD4+ Treg cells fit. Nature Imm. 6, 1071-72 (2005).

52) Fontenot, J. D., Rassmussen, J. P., Gavin, M. A. & Rudensky, A. Y. A function for interleukin-2 in FOXP3-expressing regulatory T cells. Nature Immunol. 6, 1142-1151 (2005).

53) Schmitz I. Krueger A. Baumann S. Schulze-Bergkamen H. Krammer P H. Kirchhoff S. An IL-2-dependent switch between CD95 signaling pathways sensitizes primary human T cells toward CD95-mediated activation-induced cell death. Journal of Immunology. 171(6):2930-6, 2003

54) Maher S G. . . . Toomey D M. Taurine attenuates CD3/interleukin-2-induced T cell apoptosis in an in vitro model of activation-induced cell death (AICD). Clinical & Experimental Immunology. 139(2):279-86, 2005

55) Kuge S. Watanabe K. Makino K. Tokuda Y. Mitomi T. Kawamura N. Habu S. Nishimura T. Interleukin-12 augments the generation of autologous tumor-reactive CD8+ cytotoxic T lymphocytes from tumor-infiltrating lymphocytes. Japanese Journal of Cancer Research. 86(2):135-9, 1995
56) Mehrotra P T. Wu D. Crim J A. Mostowski H S. Siegel J P. Effects of IL-12 on the generation of cytotoxic activity in human CD8+ T lymphocytes. Journal of Immunology. 151(5):2444-52, 1993
57) Barnett, T., Goebel, S. J., Nothdurft, M. A. and Elting, J. J. Carcinoembryonic antigen family: characterization of cDNAs coding for NCA and CEA and suggestion of nonrandom sequence variation in their conserved loop-domains. Genomics. 3: 59-66 (1988).

58) Bazan, J. F. Unraveling the structure of IL-2. Science. 257: 410-413 (1992).

59) Wolf, S. F., Temple, P. A., Kobayashi, M., Young, D., Dicig, M., Lowe, L., Dzialo, R., Fitz, L., Ferenz, C., Hewick, R. M. Cloning of cDNA for natural killer cell stimulatory factor, a heterodimeric cytokine with multiple biologic effects on T and natural killer cells. J. Immunol. 146:3074-3081 (1991)

60) Collaco R F. Cao X. Trempe J P. (1999) A helper virus-free packaging system for recombinant adeno-associated virus vectors. Gene. 238(2):397-405.

61) Pala, P., Hussell, T., Openshaw, P J (2000) Flow cytometric measurement of intracellular cytokines. J. Immunol. Methods 243:107-24.

62) Ghanekar S A, Nomura L E, Suni M A, Picker L J, Maecker H T, Maino V C. Gamma interferon expression in CD8(+) T cells is a marker for circulating cytotoxic T lymphocytes that recognize an HLA A2-restricted epitope of human cytomegalovirus phosphoprotein pp65. Clin Diagn Lab Immunol. 2001 May; 8(3):628-31.

63) Smith, K. A. 1988. Interleukin-2: inception, impact, and implications. Science 240: 1169-1176.

64) Sadlack, B., H. Merz, H. Schorle, A. Schimpl, A. C. Feller, I. Horak. 1993. Ulcerative colitis-like disease in mice with a disrupted interleukin-2 gene. Cell 75: 253-261.

65) Cousens, L. P., J. S. Orange, C. A. Biron. 1995. Endogenous IL-2 contributes to T cell expansion and IFN-gamma production during lymphocytic choriomeningitis virus infection. J. Immunol. 155: 5690-5699.

66) D'Souza, W. N., K. S. Schluns, D. Masopust, L. Lefrancois. 2002. Essential role for IL-2 in the regulation of antiviral extralymphoid CD8 T cell responses. J. lmmunol. 168: 5566-5572.

67) Almeida, A. R., N. Legrand, M. Papiernik, A. A. Freitas. 2002. Homeostasis of peripheral CD4+ T cells: IL-2R“TYPE=PICT;ALT={alpha}” and IL-2 shape a population of regulatory cells that controls CD4+ T cell numbers. J. Immunol. 169: 4850-4860.

68) Furtado, G. C., M. A. Curotto de Lafaille, N. Kutchukhidze, J. J. Lafaille. 2002. Interleukin 2 signaling is required for CD4+ regulatory T cell function. J. Exp. Med. 196: 851-857.

69) Curotto de Lafaille, M. A., A. C. Lino, N. Kutchukhidze, J. J. Lafaille. 2004. CD25− T cells generate CD25+Foxp3+ regulatory T cells by peripheral expansion. J. Immunol. 173: 7259-7268.

70) Leonard, W. J. 2003. Type I cytokines and interferons and their receptors. W. E. Paul, ed. Fundamental Immunology 5 Ed. 706-708. Lippincott Williams & Wilkins, Philadelphia.

71) Trinchieri, G. 1995. Interleukin-12: a proinflammatory cytokine with immunoregulatory functions that bridges innate resistance and antigen specific adaptive immunity. Annu. Rev. Immunol. 13:251.

72) Seder, R. A., R. Gazzinelli, A. Sher, W. E. Paul. 1993. IL-12 acts directly on CD4+ T cells to enhance priming for IFN-gamma production and diminishes IL-4 inhibition of such priming. Proc. Natl. Acad. Sci. USA 90:10188.

73) Hsieh, C. S., S. E. Macatonia, C. S. Tripp, S. F. Wolf, A. O'Garra, K. M. Murphy. 1993. Development of Th1 CD4+ T cells through IL-12 produced by Listeria-induced macrophages. Science 260:547.

74) Stout, R. D., J. Suttles. 1996. The many roles of CD40 in cell-mediated inflammatory responses. Immunol. Today 17:487.

75) Cella, M., D. Scheidegger, K. Palmer-Lehmann, P. Lane, A. Lanzavecchia, G. Alber. 1996. Ligation of CD40 on dendritic cells triggers production of high levels of interleukin-12 and enhances T cells stimulatory capacity: T-T help via APC activation. J. Exp. Med. 184:747.

76) Shu, U., M. Kiniwa, C. Y. Wu, C. Maliszewski, N. Vezzio, J. Hakimi, M. Gately, G. Delespesse. 1995. Activated T cells induce interleukin-12 production by monocytes via CD40-CD40 ligand interaction. Eur. J. Immunol. 25:1125.

77) Bianchi, R., U. Grohmann, M. L. Belladonna, S. Silla, F. Fallarino, E. Ayroldi, M. C. Floret, P. Puccetti. 1996. IL-12 is both required and sufficient for initiating T cell reactivity to a class I-restricted tumor peptide (P815AB) following transfer of P815AB-pulsed dendritic cells. J. Immunol. 157:1589.

78) Grohmann, U., R. Bianchi, E. Ayroldi, M. L. Belladonna, D. Surace, M. C. Fioretti, P. Puccetti. 1997. A tumor-associated and self antigen peptide presented by dendritic cells may induce T cell anergy in vivo, but IL-12 can prevent or revert the anergic state. J. Immunol. 158:3593.

79) Grohmann, U., M. C. Fioretti, R. Bianchi, M. L. Belladonna, E. Ayroldi, D. Surace, S. Silla, P. Puccetti. 1998. Dendritic cells, interleukin 12, and CD4+ lymphocytes in the initiation of class I-restricted reactivity to a tumor/self peptide. Crit. Rev. lmmunol. 18:87.

80) Bianchi, R., Grohmann, U., Vacca, C., Belladonna, M. L., Fioretti, M. C., and Puccetti, P. Autocrine IL-12 is involved in dendritic cell midulation via CD40 ligation. J. Immunology 1999, 163:2517-1521.

81) Chang H D, Radbruch A. The pro- and anti-inflammatory potential of interleukin-12. Ann NY Acad Sci. 2007 August; 1109:40-6.

82) Chang H D, Helbig C, Tykocinski L, Kreher S, Koeck J, Niesner U, Radbruch A. Expression of IL-10 in Th memory lymphocytes is conditional on IL-12 or IL-4, unless the IL-10 gene is imprinted by GATA-3. Eur J Immunol. 2007 March; 37(3):807-17.

TABLE 1

| DC treatment | CD14 | CD40 | CD80 | CD83 | CD86 | HLA-DR |
|---|---|---|---|---|---|---|
| ctrl (mock) | 22.3 | 25.9 | 45.5 | 32.9 | 68.9 | 95.4 |
| AAV/CEA | 12.6 | 45.6 | 59.8 | 46.8 | 82.4 | 98.6 |
| AAV/CEA + | 18.1 | 34.2 | 62.3 | 39.7 | 82.0 | 95.2 |
| exogen IL-2 | | | | | | |
| AAV/CEA + | 17.4 | 39.0 | 53.6 | 42.5 | 84.0 | 96.4 |
| AAV/IL-2 | | | | | | |
| AAV/CEA + | 10.7 | 50.8 | 61.8 | 53.9 | 87.8 | 97.1 |
| exogen IL-12 | | | | | | |
| AAV/CEA + | 6.5 | 66.9 | 84.6 | 65.9 | 95.7 | 97.2 |
| AAV/IL-12 | | | | | | |

TABLE 2

| DC treatment | CD8/CD4 | CD69+, CD8+ | CD25+, CD4+ |
|---|---|---|---|
| ctrl (mock) | 29.4/47.9 | 23.8 | 48.2 |
| AAV/CEA | 48.6/27.9 | 61.4 | 18.4 |
| AAV/CEA + | 17.2/39.7 | 9.7 | 29.1 |
| exogen IL-2 | | | |
| AAV/CEA + | 20.4/42.5 | 11.9 | 33.2 |
| AAV/IL-2 | | | |
| AAV/CEA + | 54.2/31.7 | 67.7 | 18.1 |
| exogen IL-12 | | | |
| AAV/CEA + | 68.0/21.5 | 80.9 | 9.2 |
| AAV/IL-12 | | | |

TABLE 3

| T cell treatment | CD8/CD4 | CD69+, CD8+ | CD25+, CD4+ |
|---|---|---|---|
| AAV/IL-2 | 35.5/18.9 | 32.2 | 43.7 |
| AAV/IL-12 | 50.2/26.8 | 61.3 | 21.1 |
| exogen IL-12 | 58.2/37.7 | 73.6 | 15.3 |

1. A method of killing problematic or disease-causing cells (such as cancer) in a patient, the method comprising, administration into the patient a therapeutically effective amount of antigen presenting cells previously treated with IL-12 gene delivery and with an antigen.

2. A method according to claim 1 wherein the IL-12 gene is administered into the antigen presenting cell by one of the following AAV virus types,1, 2, 3, 4, 5, 6, 7, 8, or 9.

3. A method according to claim 1 wherein the IL-12 gene is administered into the antigen presenting cell by one of the following virus types adenovirus, retrovirus, lentivirus.

4. A method according to claim 1 wherein the AAV/IL-12 gene is administered into the antigen presenting cell just after removal and isolation from the human donor.

5. A method according to claim 1 wherein the AAV/IL-12 gene is administered into the antigen presenting cell before treatment with IL-4 or GM-CSF.

6. A method according to claim 1 wherein the AAV/IL-12 gene is administered into the antigen presenting cell just before addition of naïve T cells.

7. A method according to claim 1 wherein the antigen is introduced into the antigen presenting cell as a gene.

8. A method according to claim 1 wherein the antigen is introduced into the antigen presenting cell as a protein.

9. A method according to claim 1 wherein the antigen delivered into the dendritic cells is a cancer antigen.

10. A method according to claim 1 wherein the antigen delivered into the dendritic cells is cell type specific antigen.

11. A method according to claim 1 wherein the antigen delivered into the dendritic cells is a viral antigen.

12. A method according to claim 1 wherein the antigen delivered into the dendritic cells is a bacterial antigen.

13. A method according to claim 1 wherein the antigen delivered into the dendritic cells is a parasitic antigen.

14. A method according to claim 9 wherein the cancer is selected from the group consisting of prostate cancer, breast cancer; bladder cancer; lung cancer; thyroid cancer; leukemias; lymphomas; colon cancer; glioma; liver cancer; pancreatic cancer; renal cancer; cervical cancer; testicular cancer; head and neck cancer; ovarian cancer; neuroblastoma and melanoma.

15. A method according to claim 10 wherein the cell is selected from the group consisting of hyperplasia, fibrosis, and carcinoid.

16. A method according to claim 11 wherein the virus is selected from a group comprising of herpes virus, adenovirus, pox virus, papillomavirus, polyomavirus, influenza, human immunodeficiency virus, ebola virus, hepatitis C virus, hepatitis B virus.

17. A method of killing problematic or disease-causing cells in a patient, the method comprising, administration into1. A method of killing problematic or disease-causing cells in a patient, the method comprising, administration into the patient a therapeutically effective amount of cytotoxic T lymphocytes (CTL), wherein the CTLs have been generated by coincubation with antigen presenting cells previously treated with AAV/IL-12 gene delivery and with an antigen.
the patient a therapeutically effective amount of cytotoxic T lymphocytes (CTL), wherein the CTLs have been generated by coincubation with antigen presenting cells previously treated with IL-12 gene delivery and with an antigen.

18. A method according to claim 17 wherein the IL-12 gene is administered into the antigen presenting cell by one of the following AAV virus types,1, 2, 3, 4, 5, 6, 7, 8, 8, or 9.

19. A method according to claim 17 wherein the IL-12 gene is administered into the antigen presenting cell by one of the following virus types adenovirus, retrovirus, lentivirus.

20. A method according to claim 17 wherein the AAV/IL-12 gene is administered into the antigen presenting cell just after removal and isolation from the human donor.

21. A method according to claim 17 wherein the AAV/IL-12 gene is administered into the antigen presenting cell before treatment with IL-4 or GM-CSF.

22. A method according to claim 17 wherein the AAV/IL-12 gene is administered into the antigen presenting cell just before addition of naïve T cells.

23. A method according to claim 17 wherein the antigen is introduced into the antigen presenting cell as a gene.

24. A method according to claim 17 wherein the antigen is introduced into the antigen presenting cell as a protein.

25. A method according to claim 17 wherein the CTL target cancer antigens.

26. A method according to claim 17 wherein the CTL target cell type specific antigens.

27. A method according to claim 17 wherein the CTL target viral antigens.

28. A method according to claim 17 wherein the CTL target bacterial antigens.

29. A method according to claim 16 wherein the CTL target parasitic antigens.

30. A method according to claim 23 wherein the cancer is selected from the group consisting of prostate cancer, breast cancer; bladder cancer; lung cancer; thyroid cancer; leukemias; lymphomas; colon cancer; glioma; liver cancer; pancreatic cancer; renal cancer; cervical cancer; testicular cancer; head and neck cancer;
ovarian cancer; neuroblastoma and melanoma.

31. A method according to claim 24 wherein the cell is selected from the group consisting of hyperplasia, fibrosis, and carcinoid.

32. A method according to claim 25 wherein the virus is selected from a group comprising of herpes virus, adenovirus, pox virus, papillomavirus, polyomavirus, influenza, human immunodeficiency virus, ebola virus, hepatitis C virus, hepatitis B virus.


SEQUENCE LISTING (8 sequences)

SEQ ID NO: 1 - DNA, 18 bp, Human
accatggagtctccctcg

SEQ ID NO: 2 - DNA, 18 bp, Human
ctatatcagagcaacccc

SEQ ID NO: 3 - DNA, 20 bp, Human
tgccacaatgtcaccagtat

SEQ ID NO: 4 - DNA, 21 bp, Human
cggagtatacttatatgacgc

SEQ ID NO: 5 - DNA, 18 bp, Human
aatgtggccccctgggtc

SEQ ID NO: 6 - DNA, 18 bp, Human
tttaggaagcattcagat

SEQ ID NO: 7 - DNA, 20 bp, Human
agatgtgtcaccagcagttg

SEQ ID NO: 8 - DNA, 18 bp, Human
aacctaactgcagggcac